(19) United States
(12) Patent Application Publication
(10) Pub. No.: US 2007/0149450 A1
(43) Pub. Date: Jun. 28, 2007

(54) Method for reducing levels of c-reactive protein

(76) Inventors: Ranjit Bhardwaj, Berlin (DE); Birgit Vogt, Berlin (DE); Ahmed Sheriff, Berlin (DE)

Correspondence Address: JACOBSON HOLMAN PLLC, 400 SEVENTH STREET N.W., SUITE 600, WASHINGTON, DC, 20004, US

(21) Appl. No.: 10/546,728

(22) PCT Filed: Feb. 26, 2004
(86) PCT No.: PCT/EP04/01915

§ 371 Date: Jun. 26, 2006

(30) Foreign Application Priority Data
Feb. 27, 2003 (EP) 03004444.

(51) Int. Cl. A61K 38/17; C07K 14/705

(52) U.S. Cl. 514/12; 530/350

(57) ABSTRACT

A compound comprising at least a structural entity which binds C-reactive protein (CRP) or parts of it or CRP in its monomeric, pentameric or multimeric form, preferably human CRP and which a) blocks one or more CRP functions on cell surfaces or in a solution, preferably blood or other body fluids or from tissues, most preferably in vivo, b.) and/or depletes CRP from a solution, preferably blood or other body fluids or from tissues, most preferably in vivo.

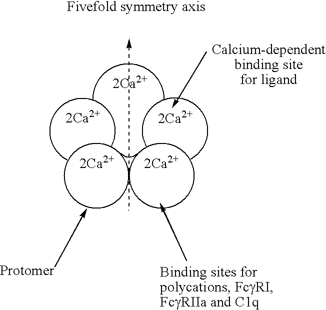

The current invention relates to a method for decreasing levels of C-reactive protein (CRP) in humans comprising administering to a mammal in need thereof an effective amount of a compound containing a molecule that binds CRP or a pharmaceutical salt or solvate thereof.

### BACKGROUND OF THE INVENTION

C-reactive protein (CRP) is an acute-phase constituent with a record of service for more than seven decades. In the last decade, the protein experienced a revival in attention due to the inflammatory pathogenesis of atherosclerosis. In particular, the role of CRP in the vulnerability and instability of atherosclerotic plaques, leading to rupture, thrombosis and thus to occlusive arterial disease, has been studied.

C-reactive protein (CRP) is the prototype acute-phase protein, which can increase up to 1000-fold after the onset of a stimulus. Aside from its disputed role as a marker of infection and/or inflammation in daily clinical practice, the protein has a wide variety of biological properties and, functions. Due to its opsonizing abilities and its capability to activate human complement, CRP plays an important role in the innate host defense against different microorganisms, such as bacteria and fungi. The same opsonophagocyting properties can lead to clearance of host cell material, including nuclear constituents. Inflammation is one of the cornerstones in the etiology and pathogenesis of atherosclerosis, which led to, worldwide attention being focused on CRP and its role in the process of atherosclerosis. This role may have a dual character. First, CRP levels reflect the ‘burden’ of inflammation within atherosclerotic lesions, thus reflecting the grade of vulnerability and instability of the plaques. For this reason, an Increased level of the protein may be a prelude to rupture of the plaque and, thus, to occlusive arterial disease. Secondly, CRP may play an active role in the atherosclerotic process. CRP plays a role in the expression of different adhesion molecules on endothelial cells and the protein is able to activate human complement within the plaque. Furthermore, the recent discovery of local production of CRP and complement proteins within the plaque suggests an active role for the protein in the inflammatory cascade. Whatever the role for CRP in the atherosclerotic process, it has been proven that an elevated CRP level, with a cut-off point of approximately 3 mg/l, is associated with an increased risk of occlusive arterial disease, especially acute coronary syndromes.

History of CRP: Fundamentals and Clinical Use

Historical Perspectives

C-reactive protein (CRP) is an acute-phase protein that was discovered in 1930 by William S. Tillet and Thomas Francis at the Rockefeller Institute for Medical Research, 3 Exp Med 1930; 52:561-571.

CRP was the first of a scala of proteins that was found in the acute phase of an infection. The concentrations of these proteins increased to as much as 1000-fold (CRP). The acute-phase response, i.e. the changes in concentrations of the acute-phase proteins, is a non-specific innate defense mechanism of the host. There are many other conditions besides bacterial infections that lead to an acute-phase response including inflammation, necrosis, malignancies, burns, surgery, trauma,-childbirth, strenuous exercise, stress, and psychiatric disease.

Structure and Binding Sites of CRP

CRP is a protein of the highly conserved pentraxin family with a striking sequence homology between species going back as far as the horseshoe crab. Another striking feature is the lack of polymorphism within a species. It is built up of five identical subunits (protomers) aggregated in a symmetric pentameric form by noncovalent binding between the subunits. Each subunit consists of 206 amino acids in a single polypeptide chain with a total molecular weight of approximately 23 000 Da. The two cystine residues at positions 36 and 78 realise a disulfide bond. Each subunit has the ability to bind two calcium ions so that a calcium-dependent specific binding of a ligand is possible. The most avid ligand is phosphocholine (PCh), a constituent of the phospholipids of cell membranes and plasma lipoproteins. Phosphocholine is universal for most eukaryotic organisms. Fraction C of the bacterial cell wall, as described above, also contains phosphocholine. Due to conformational changes in the CRP macromolecule, the binding affinity of each consecutive ligand is highly increased. In other words, CRP is an allosteric protein.

Other ligands of the calcium-dependent binding property of CRP are nuclear constituents: histones, chromatin, and small nuclear ribonucleoproteins (snRNPs). Therefore, CRP may play an important role in the clearance and processing of nuclear antigens, thus preventing autoimmune responses to nuclear material. A schematic illustration of CRP is depicted in FIG. 1.

A calcium-independent binding exists for cationic polymers (lysine- and arginine-rich proteins). The role of this binding property may be that it has modulatory effects on the inflammatory process because most polycations are secreted by neutrophils.

2. 3. Biosynthesis and Kinetics of CRP

The major site of CRP synthesis is the hepatocyte. Under physiological circumstances, human CRP is a protein with a median serum concentration of 0.8 mg/l. The human CRP gene is located on the long arm of chromosome 1. Plasma CRP is mainly regulated at the transcriptional level induced by IL-6. In vitro and in vivo CRP mRNA transcription is dramatically upregulated by IL-6. This response is greatly enhanced in combination with IL-1b. This synergistic phenomenon occurs due to the regulation of CRP synthesis at the translational level by IL-1b. After transcription, CRP mRNA is translated to protomers. In the endoplasmic reticulum five protomers are assembled to one cyclic pentamer, which is either secreted or stored.

When released in the circulation, the protein is equally distributed in the vascular compartment without substantial tissue sequestration at sites of inflammation. This could be explained by the ‘detoxification’ hypothesis: binding and thereby neutralizing/detoxifying harmful substances that escape from the site of inflammation to the circulation. The dramatic rise in CRP levels may exceed 300 mg/l within 48 h after the acute event. High levels may persist during the presence of the stimulus. There is a strong positive correlation between the duration and the intensity of the stimulus (e.g. tissue injury) and the number of hepatocytes synthesizing CRP. The latter phenomenon is due to the activation of the hepatocytes in the direction of the blood flow: cytokines first arrive at the hepatocytes in the vicinity of the portal triangle and further activation of the hepatocytes takes place in the direction of the central vein. This results in a higher peak level and also a protracted increase in serum CRP whenever the degree of the stimulus is stronger and longer. Most of the CRP is taken up and degraded at the same site of production: the hepatocyte. A small part (bound to its ligands) is taken up and processed by neutrophils and macrophages.

Clearance of the protein from the bloodstream is mono-exponential with a biological half-time of 19 h. This half-time is independent of the CRP level and, therefore, independent of the physiological or pathophysiological circumstances. Thus, the only significant determinant of plasma CRP levels is the rate of synthesis, which in principal justifies the clinical use of serum CRP to monitor the activity of the inflammation or other disease process.

Post-transcriptional mechanisms also play a role in the CRP level. After a stimulus there is a pronounced acceleration of the secretion of CRP from the endoplasmic reticulum, explaining the rapid rise in concentration.

Previous investigators also identified extrahepatic generation of CRP: in brain neurons in patients with Alzheimer's disease, a minority of peripheral. lymphocytes, and within atherosclerotic plaques. The latter may play an important role in the inflammatory process of atherosclerotic lesions.

Biological Properties and Function of CRP

The biological functions of CRP are diverse and can be derived from its binding properties. These are summarized in Table 1.

The calcium-dependent binding of CRP to phosphocholine results in a CRP-Ca2+-PCh complex. This ligand-complexed CRP is recognized by C1q and leads to the formation of C3 convertase and, thus, to activation of the classical pathway of the human complement. The activation of the classical pathway leads to opsonization and phagocytosis of phosphocholine-containing microorganisms via the terminal membrane attack complex (FIG. 2). The processing and clearance of necrotic host cell material is done via the same route: calcium-dependent binding of nuclear material or other cell material to CRP leads to the activation of the classical pathway of complement and, thus, to opsonophagocytosis. However, for apoptotic host cell material, the last route, the terminal membrane attack complex, is not activated. In this way, the apoptotic host cell material is elegantly cleared without further inflammatory damage.

Another important biological property is the ability of ligand-complexed CRP to bind to the FcgRI and FcgRIIa receptors (Fc receptors for IgG molecules). This binding elicits a response of phagocytic cells and thus enhances the phagocytosis of microorganisms or damaged/dead host cell material (FIG. 3).

TABLE 1
Biological properties and functions of CRP

| Biological property | Anion | Function |
|---|---|---|
| Calcium-dependent binding of phosphocholin and other phosphate esters | 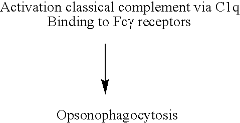 | Innate host defense against inferoorganisms (bacteria, fungi) |
| Calcium-dependent binding of histones, snRNP, chromatin | 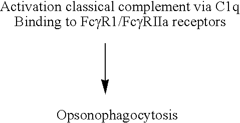 | Non inflammatory clearance/processing of nuclear host material |
| Calcium-dependent binding of cellular host material | 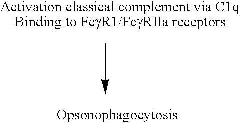 | Non-inflammatory clearance/processing apopiotic cells |
| Binding of polycations | Modulatory effect on neutrophils | Modulatory effect on the |
| | | inflammatory process |
| Expression of ICAM-1, VCAM-1 and | Enhancing of adhesion and recruitment | Enhancing vascular wall inflammation |
| E-selection on endothelial cells by | of monocytes and lymphocytes | |
| mCRP | | |
| Binding of CRP/mCRP | Chedding L-selectin on neutrophils | Prevention of adhesion of neutrophils |
| to FcγRIIIb receptors of neutrophils | | to endothelial cell |

ICAM-1, Intercellular adhesion molecule-1; VCAM-1, vascular cell adhesion molecule-1; mCRP, modified CRP.

Table 1 (Gewurz, H.; Zhang, X. H.; Lint, T. F.; Curr Opin Immunol, 1995; 7:54-64)

Some of the pentameric CRP molecules undergo processes of proteolysis or denaturization. The first process results in dissociation of pentameric CRP into monomeric subunits or smaller peptides. Conformational changes of the molecule due to denaturization lead to modified CRP molecules (mCRP). The final result of both processes is the expression of new epitopes, called neoepitopes, which normally are ‘hidden’ in the native molecule. Different functions are attributed to the distinct binding properties of the neoepitopes from native CRP. For example, a third binding facility of mCRP is to the low-affinity IgG receptor FcgRIIIb on the neutrophil. This binding results in shedding of L-selectin and, thus, inhibition of adhesion of the neutrophil to the endothelial cell. This antiinflammatory effect of mCRP may play a role in the fact that neutrophils are absent in atherosclerotic lesions.

Clinical Use of C-Reactive Protein

As stated above, clearance of CRP is monoexponential and independent of the serum concentration or pathophysiological circumstances. Therefore, measurement of CRP is a good marker of disease activity. However, the use of CRP in the diagnostic process of the physician is controversial. The potential clinical use of CRP measurements has been appreciated by some and rejected by others.

The use of CRP to distinguish viral from bacterial infections remains a matter of dispute, at the very least. Though very high levels of CRP tend to be associated with invasive bacterial infection, previous reports have also shown CRP levels of 100 mg/l or higher in acute viral infections. The acute-phase response probably depends on the extent of the host tissue damage caused by -invasive viruses or by the host immune response to the virus with due tissue damage. Furthermore, CRP levels in the acute phase of a bacterial infection can still be normal in the first 24 h after the onset of the infection. In conclusion, CRP measurements can only be interpreted in concert with other clinical and laboratory information.

CRP has proven to be an objective measure of disease activity in rheumatoid arthritis (RA) and is useful to the physician for monitoring effects of drug therapy in this disease. Moreover, persistent high levels of CRP are a risk factor for continuing joint deterioration. There are a few diseases with relatively low or normal CRP levels during disease activity: systemic lupus erythematosus (SLE), polymyositis, primary Sjogren's syndrome, acute leukemia, and ulcerative colitis.

Some clinicians use plasma CRP concentrations to differentiate between a SLE exacerbation and intercurrent infections. However, in SLE exacerbations accompanied by serositis, median CRP levels are comparable to the CRP levels measured during infections. This is also the case for SLE accompanied by polyarthritis, though to a lesser degree. The cause of the poor CRP response is unknown. Serologic studies have shown that there is an adequate IL-6/IL-1/TNF-60 response during disease activity.

The tendency to find greater increases in circulating CRP in patients with active Crohn's disease compared to ulcerative colitis is attributed to the production of higher amounts of IL-1b and Il-6 by monocytes and macrophages in patients with Crohn's disease. The difference, however, is not significant enough to use the plasma CRP concentration in the differential diagnosis, let alone disease activity.

Revival of CRP: Atherosclerosis and Inflammation

Atherosclerosis and Plaque Formation

Atherosclerosis is a slowly progressive disease that begins in early childhood and smolders until it becomes manifest at middle age or later by a cardiac event, stroke, or peripheral vascular disease. The first macroscopic stadium of atherosclerosis is the fatty streak, a slightly elevated yellow lesion within the intima, microscopically filled with foam cells (macrophages loaded with cholesterol esters and free cholesterol), smooth muscle cells, and a few T lymphocytes. In a later stage, the fatty streak may develop to an atheromatous or fibrous plaque. A typical plaque of the later phase is characterized by: (i) a fibrous cap bordering the plaque on the luminal side, containing connective tissue with smooth muscle cells; (ii) a cellular area beneath the fibrous cap, consisting of macrophages, smooth muscle cells, and T lymphocytes; and (iii) a deeper necrotic core, containing cellular debris, lipids, cholesterol crystals, and calcium deposits.

The relative contents of fibrous tissue, lipids, cells, and calcium deposits may vary. Like the contents of the plaque, the plaque itself may also vary in a spectrum from atheromatous, fibrous, to calcified plaques, with such possible complications as thrombosis, ulceration, or rupture.

The etiology and pathogenesis of atherosclerosis and plaque formation is not understood in detail. Though hyperlipidemia plays an important role in the progression of atheromatous plaques, the fact that fatty streaks appear very early in life suggests that other etiological factors also play a crucial role. Fatty streaks appear as early as in fetal life, mainly in the thoracic aorta, near the region of the aortic valve ring, and increase in number and involved area until the third decade of life. The process of fatty streak formation in the unborn is greatly enhanced by maternal hypercholesterolemia, but they also appear in fetuses of normocholesterolemic mothers. From the second decade on, the other major arteries also become involved. Most of the fatty streaks remain unchanged or even disappear in life. Not until adolescence will a progressive development of atheromatous plaques appear. Preferential sites in the arteries where fatty streaks may further develop to plaques seem to be at locations where alterations in the dynamics of blood flow appear: branches, bifurcations, and curvatures.

The central question to be answered is: what is the first step in atherosclerosis? The most recent response-to-injury hypothesis states that endothelial dysfunction is the second step in atherosclerosis. But what precedes endothelial dysfunction? What is the nature of the injury? Several etiological factors have been proposed: elevated and modified LDL, smoking, hypertension, hyperhomocysteinemia, diabetes mellitus, infections (Chlamydia pneumoniae, herpes viruses, CMV, Helicobacter pylon), and anti-beta2 glycoprotein I antibodies. The process may be multifactorially determined, in which case genetic factors also play an important role.

Endothelial dysfunction leads to expression of different adhesion molecules (ICAM-1, VCAM-1, P-selectin and E-selectin) on the endothelial cells, thus promoting adhesion and ‘rolling’ of monocytes and T lymphocytes. The expression of these molecules is enhanced in areas of turbulent flow with low shear stress. After transmigration of monocytes they become macrophages and take up lipids (oxidized LDL) via scavenger receptors, forming foam cells.

In time the foam cells die and the cellular debris and the lipids form the constituents of the necrotic core underlying the fibrous cap.

The increased permeability of the endothelium and the production of cytokines by the endothelial cells enhance the process of adhesion and migration. After the invasion of the inflammatory cells within the vascular wall, they produce different cytokines, which results in migration and proliferation of smooth muscle cells (SMCs) from the medial layer. The fibrous tissue (collagens and elastines) production by the SMCs forms the cement of the fibrous cap, whereas the cells can be considered the bricks. The above is illustrated in FIG. 4.

Stable and Unstable Plaques: Vulnerability and the Process of Destabilization

Histopathological studies have revealed that the majority of acute coronary events are caused by plaque rupture with ensuing thrombus formation on the highly thrombogenic mass lying beneath the ruptured fibrous cap. They have also found that the vulnerable plaques prone to rupture have a large lipid pool, are bordered by a thin fibrous cap, and cause only moderate to mild stenosis of the arterial lumen. The majority of plaque ruptures occur at the shoulder region of the fibrous cap. In this region, there are three striking features: an accumulation of inflammatory cells (monocytes/macrophages and T lymphocytes), expression of matrix-degrading metalloproteinases (MMPs), and a higher circumferential tensile stress due to plaque configuration and composition. The concert of these three features may result in progressive necrosis of the lipid core and thinning of the fibrous cap, resulting in a vulnerable and unstable plaque. For example, the over expression of MMPs in the shoulder region may be part of a remodelling process induced by the high tensile stresses occurring in these regions. The accumulation of inflammatory cells led to the inflammation hypothesis.

Atherosclerosis and Inflammation : the Role of C-Reactive Protein

The most convincing evidence that atherosclerosis is an inflammatory process, and not merely a process of depositions of lipids In the arterial wall, is the continuous presence and accumulation of monocyte-derived Macrophages and T lymphocytes in fatty streaks and advanced atherosclerotic lesions. There is also evidence that activated complement and CRP are present in atherosclerotic lesions. CRP is found to bind the phosphocholin group of enzymatically degraded, nonoxidized LDL (E-LDL) within early atherosclerotic lesions. The E-LDL-Ca -CRP complex is internalized by the macrophages and activates complement via the classical route and enhances the inflammatory process.

Thus, atherosclerosis can be considered to be a chronic low-grade inflammatory disease with a continuous low grade production of pro-inflammatory mediators by T lymphocytes and macrophages: TNF-a; IL-1, and IL-6.

These cytokines escape from the plaque into the circulation. The increased cellular infiltration of coronary plaques in patients with unstable angina pectoris results in a higher plateau of the chronic inflammatory process: an increased production of pro-inflammatory cytokines. This results in an increased production in the liver of the inflammatory proteins CRP, serum amyloid P component, serum amyloid A, and fibrinogen.

This inflammatory profile is more pronounced when the atherosclerotic disease is more advanced. For example, patients with both coronary disease and peripheral arterial disease have higher plasma levels of the acute-phase proteins than patients presenting with only one of the disease states.

CRP, the prototype acute-phase protein, is the most rapid responder after an inflammatory stimulus and may be a good candidate for a marker of the inflammatory part of plaque formation. To study the role of CRP, a highly sensitive assay is required because the magnitude of concentration differences that are postulated to play a role in the pathogenesis of atherosclerosis lies in the order of tenths of milligrams per liter.

Besides CRP being a marker of the inflammatory process, CRP may also play a role as an inducer of the inflammatory process. CRP and modified CRP play a modulating role at the, site of inflammation by their effect on the expression of different adhesion molecules. For example, the set of inflammatory cells found within the plaque may be mirroring the effect of CRP/mCRP: inhibition of neutrophil adhesion and the stimulation of monocyte and T lymphocyte adhesion to the endothelium.

Both CRP and mCRP are found in human blood vessels, and it would therefore be plausible to ascribe an active role for CRP/mCRP in atherogenesis. There are a few other arguments in favor of CRP being one of the engines of the inflammatory cascade. First, as mentioned earlier, the extracellular deposition of E-LDL within early atherosclerotic plaques provokes complement activation (C1-C9) when bound to CRP. The activation of the membrane attack complex enhances further inflammation. Secondly, elevated levels of CRP are associated with obesity and insulin resistance. Adipose tissue is an important source of IL-6 and TNF-a in healthy subjects and is responsible for approximately 30% of the systemic IL-6 and TNF-a. Thus, a major portion of circulating CRP, with its possible deleterious effects on the vascular wall, is indirectly caused by adipose tissue. The production of the above pro-inflammatory cytokines may play a role in the insulin resistance syndrome in obese subjects. Thirdly, a group of investigators recently discovered local production of CRP and complement proteins by macrophages and probably SMCs in arterial tissue. Using reverse transcriptase-polymerase chain reaction techniques, they found mRNAs of CRP and classical complement proteins (C1-C9, thus including the membrane attack complex) within atherosclerotic lesions, indicating local production. This could be evidence of a self-perpetuating inflammatory process within the lesion, leading to the instability and rupture of the plaque. Fourthly, CRP stimulates different cells to produce pro-inflammatory factors: the release of IL-1b, IL-6, and TNF-a by monocytes and the expression of ICAM-1 and VCAM-1 and monocyte chemotactic factor (MCP-1) by endothelial cells.

The Utility of CRP in Atherosclerotic Risk Assessment

The consensus that atherosclerosis is, at least in part, an inflammatory disease led to the question of whether there might be other screening methods in which inflammatory markers play a role. Since 1994, numerous studies have been published about the role of CRP in atherosclerotic disease. Cholesterol screening only identifies half of the individuals who are at an increased risk of developing an acute vascular event. The other half of the individuals presenting with a coronary event, stroke, or peripheral arterial disease have normal cholesterol levels and are, therefore, difficult to identify for primary prevention strategies.

Previous studies have already reported elevated CRP levels in patients with unstable angina (UA). In 1994, Luzio et al. for the first time proved the prognostic value of CRP in patients with UA. In this study, patients with UA and a CRP level above 3.0 mg/l were at a greater risk of developing a cardiac event (myocardial infarction, the need for coronary vascularization, or cardiac death). In later studies, investigators also found the prognostic relevance of CRP measurements for future risk assessment of a first cardiac event in initially healthy, middle-aged men and postmenopausal women. Thus, CRP measurements could play an important role as an independent risk factor for future coronary events.

The quintile approach would be a useful tool for risk estimations for future cardiovascular events. Subjects can be classified into five groups: quintile 1 to quintile 5. The 1st quintile, with the lowest range of hsCRP, represents subjects with the lowest risk, whereas the 5th quintile, with the highest range of hsCRP, represents subjects with the highest risk. An even stronger predictive power is obtained when the two independent risk factors are combined: CRP and the atherogenic index (ratio TC:HDL-C). The above is exemplified in Table 2. The quartile approach is based on the same principle. Thus, the additive effect of measuring hsCRP in combination with lipid measurements could result in a strong risk assessment in primary prevention as well as in secondary prevention.

TABLE 2
Atherosclerotic risk assessment (relative risks) using quintiles

| | | Quintile | | | | |
|---|---|---|---|---|---|---|
| | | 1 | 2 | 3 | 4 | 5 |
| | | hsCRP range [mg/l] | | | | |
| Quintile | TC:HDL-C | <0.7 | 0.7-1.1 | 1.2-1.9 | 2.0-3.8 | >3.9 |
| 1 | <3.4 | 1 | 1.2 | 1.4 | 1.7 | 2.2 |
| 2 | 3.4-4.0 | 1.4 | 1.7 | 2.1 | 2.5 | 3 |
| 3 | 4.1-4.7 | 2 | 2.5 | 2.9 | 3.5 | 4.2 |
| 4 | 4.8-5.8 | 2.9 | 3.5 | 4.2 | 5.1 | 6 |
| 5 | >5.8 | 4.2 | 5 | 6 | 7.2 | 8.7 |

hsCRP, high sensitivity CRP; TC:HDL-C, ratio of total cholesterol and HDL cholesterol. Data from Rifai and Ridker [55].
(Clin. Chem. 2001, 47: 403-11)

A New Target in Atherosclerotic Disease: Lowering CRP Levels

The inflammatory pathogenesis of atherosclerotic disease would seem to indicate that CRP measurements and the goal of lowering CRP levels may be tools in the-treatment strategy of the physician. Below, we will discuss the different possibilities with regard to primary and secondary prevention.

Statins

HMG-CoA reductase inhibitors (statins) are extensively used as lipid-lowering drugs in patients with atherosclerosis. For reasons of cost-effectiveness, statin therapy for primary prevention is restricted to patients with overt hyperlipidemia. However, the clinical and pathological effects of statins cannot be attributed to the lowering of circulating LDL alone. Among their other effects (antithrombotic, anti-oxidant), statins may also have anti-inflammatory properties. Several studies have demonstrated the CRP-lowering capacity of statins, independent of changes in lipid profiles. Moreover, the effect of statins is more profound in patients with CRP levels in the highest quartiles or quintiles in combination with low lipid levels.

To distinguish high-risk from low-risk patients, CRP measurements may be a good target in monitoring the effectiveness of statins. This may also have implications for the use of statins in primary prevention in patients without hyperlipidemia. Aspirin

Aspirin may be considered primarily a platelet aggregation inhibitor, but it also has anti-inflammatory actions. For many years it has proven its benefit in the primary and secondary prevention of myocardial infarction and ischemic stroke. In 1997, Ridker et al. (New Engl. J. Med. 1997; 336:973-9) demonstrated that the reduction in risk of myocardial infarction in patients using aspirin was directly correlated with the decrease in CRP level. A later study confirmed this effect (Kennon S., et al.; J Am Coll Cardiol 2001; 37:1266-70) However, in the same study, the investigators also found that the predictive value of CRP levels as a risk factor for a coronary event in patients presenting with UA was lost after pretreatment with aspirin. Furthermore, the extent of tissue damage after myocardial infarction was also reduced in patients who were pretreated with aspirin. The latter effect was possibly due to the anti-inflammatory property of the drug. Thus, the protective effect of aspirin in primary and secondary prevention of acute arterial events may be partly due to the anti-inflammatory properties of the drug.

Antibiotics

Sero-epidemiological studies have shown an increased incidence of infections with Cytomegalovirus, Herpes simplex virus 1, Helicobacter pylori, and Chlamydia pneumoniae in patients with atherosclerotic disease.

The strongest association between atherosclerotic disease and an infectious agent has been found for C. pneumoniae. Moreover, C. pneumoniae has also been detected in atherosclerotic plaques of endarteriectomic specimens in up to 30% of subjects when identified by DNA-PCR techniques. Whether infectious agents play a role in the etiology or pathogenesis of atherosclerosis or whether they are just innocent bystanders is as yet unclear. However, the possible role of microbes as causative agents in atherothrombosis has led to the idea that antibiotics may play a role in the treatment of acute arterial events.

C. pneumoniae are bacteria that infect endothelial cells, SMCs, and macrophages, cause chronic and recurrent infections, and reproduce, survive, and travel within macrophages. It is assumed that, after a mostly asymptomatic respiratory tract infection, the organisms enter the artery via the vasa-vasorum, after which SMCs are the first cells to be infected. In addition to the pathological and epidemiological findings, these properties make Chlamydia the microorganism most consistently associated with plaque formation, making it a good candidate for antibiotic targeting.

However, several retrospective and prospective studies (Kou, C. et al., J Infect Dis 2000, Suppl 3:5432-6) with short courses (1 week) and long courses (up to 3 months) of antibiotics give conflicting data on the effectiveness of antibiotics in atherothrombotic disease. It should be stated that patients who were identified as subjects with possible chlamydial infection had positive IgG serology, but it is not known whether these patients actually had an active chlamydial infection. Furthermore, it is not known if antibiotic treatment eradicates the chlamydial infection within the plaque, a difficult or even impossible investigatory task in human subjects.

In a recent study (Johnston, C. S., et al., Stroke 2001; 32:2748-52), investigators found a positive association between the identification of viable C. pneumoniae (positive RNA-PCR) within plaques and high CRP levels. This could be explained by the fact that some atherosclerotic plaques are indeed invaded by the microbe and cause an active infection with increased vulnerability of the plaque. Measuring plasma CRP, however, cannot discriminate between patients with viable Chlamydia and patients with only positive serological tests. These possible high-risk patients can only be identified via tissue research. Prospective studies should be performed to investigate the effect of antibiotic treatment on this group of patients. As yet, the use of antibiotics in primary and secondary prevention of atherothrombotic disease is not warranted. D. Thompson et al. in STRUCTURE, vol.7, no. 2, Jan. 27, 1999 (1999-01-27), pages 169-169-177, discloses that the tissue injury-enhancing effect of CRP, as a complication after atherosclerosis, may be inhibited, the latter being an attractive therapeutic target. The relevance of the development and refinement of inhibitors of CPR binding for use as drugs is recognized.

WO-A-90/12632 discloses the extracorporeal treatment of blood plasma from a cancer patient, by using an absorbent matrix including phosphorylcholine, so as to remove CRP.

JP 62 036399 A; (Nippon Biotest Kenk), Feb. 17, 1987, (1987-02-17); discloses the recovery and purification of human CRP using absorption chromatography with anti-human CPR mAb CPB 18.

W. Nunomura et al.; Journal of Biochemical and Biophysical Methods, vol. 21, no. 1; discloses the purification of human CRP by immunoaffinity chromatography using mouse mAb.

H. Jiang et al.; The Journal of Immunology, vol. 146, no. 7, pp. 2324-2330; discloses mAb 8D8 which binds to CRP and which blocks binding of CRP to the complement component C1q and which blocks CRP-induced complement activation.

R. Christner et al.; Archives of Biochemistry and Biophysics, vol. 314, no. 2, pp. 337-343; discloses mAb Ea4-1 which binds to CRP and inhibits binding of CRP to PC. MAb 1C12 binds to SAP and apparently also to CRP, and inhibits binding of CRP to PC.

S. Swanson et al.; Journal of Cellular Biochemistry, vol. 40, no. 1, 1989, pp. 121-132; discloses that CRP-laminin interaction may be blocked by mAbs that bind to CRP.

B. Hansen et al.; The Faseb Journal; vol. 3, no. 3, 1989, page A820; discloses the differential inhibition of normal and activated NK cells by antibodies against CRP.

C. Labarrere et at.; The Lancet, vol. 360, no. 9344, 2002, pp. 146Z-1467; discloses that reduction of the increased serum CRP concentrations might have beneficial effects on the rate of development and progression of transplant coronary artery disease as well as allograft failure.

### FIGURE LEGENDS

FIG. 1. Schematic illustration of pentameric CRP with possible binding sites.

FIG. 2. Processing and clearance of host cell material and invaders. Ligands of the bacterial or fungal cell wall or from host cell material (including nuclear constituents) bind to the pentameric CRP in a calcium-dependent way. Opsonization and phagocytosis is effected via two routes: the activation of the classical pathway of the human complement and through direct binding to IgG receptors.

FIG. 3. A bacterium is opsonized by CRP molecules by the calcium-dependent binding of the molecule to the phosphocholine group of the cell membrane. The bacterium is attached to the phagocytic cell by the Fcgamma-receptor binding of CRP on the phagocytic cell. After opsonization and attachment, the bacterium is internalized and phagocyted.

FIG. 4. A schematic and partly hypothetical model for the process of atherosclerosis and plaque formation.

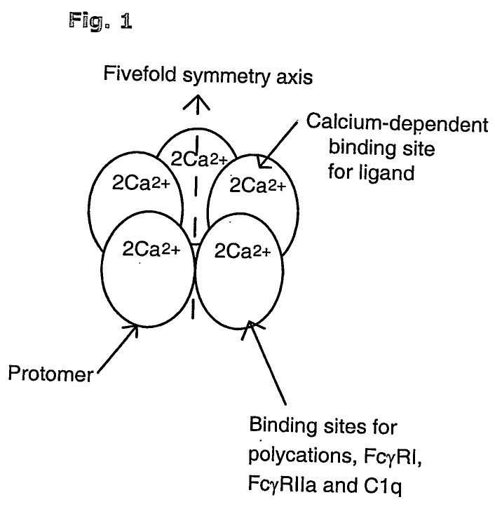

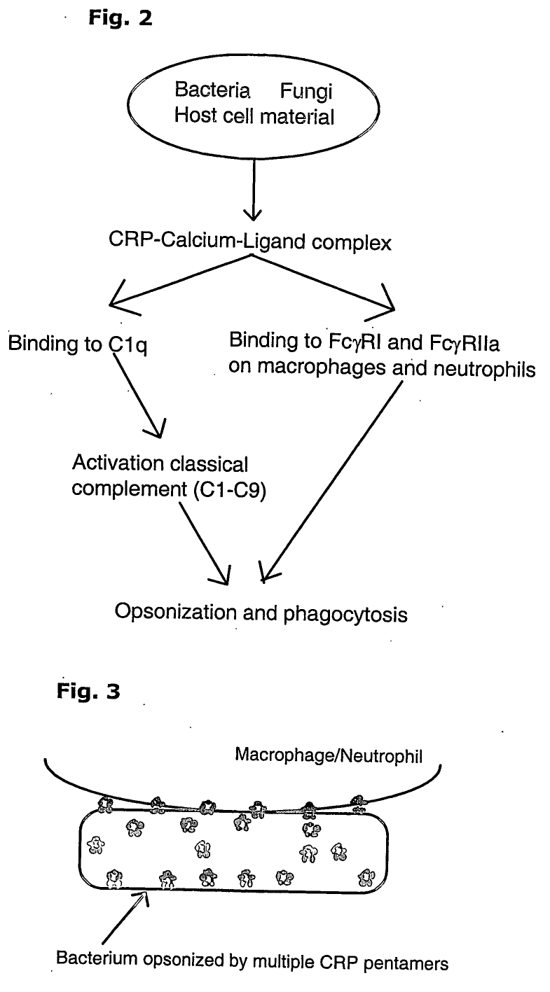

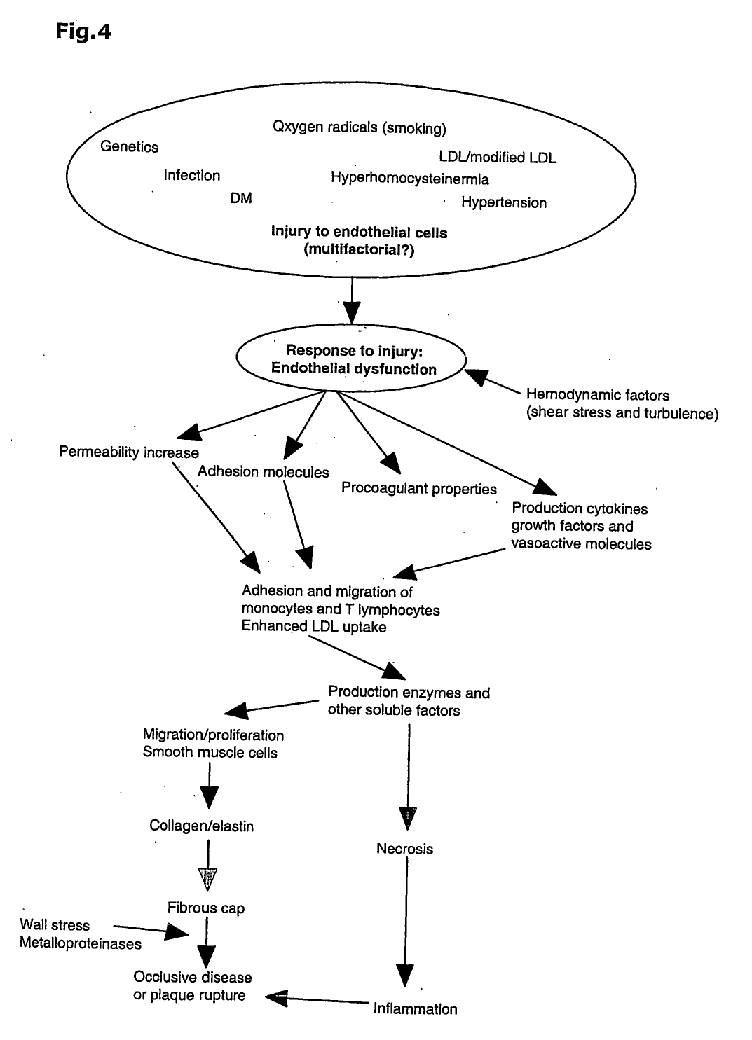

### FIELD OF INVENTION

The present invention deals with the disciplines of therapeutic proteins, cardiovascular physiology, and pharmacology. Specifically, the present invention is related to decreasing known risk factors of e.g. cardiovascular disease and other related diseases with endothelial participation associated with increased levels of C-reactive protein (CRP) by administering molecules that bind CRP.

Cardiovascular disease is a major cause of death in the United States and a major source of morbidity, medical cost, and economic loss to millions of people. Two of the most common and destructive aspects of cardiovascular disease are the appearance of arteriosclerosis and thrombolitic events.

In recent years, a great deal of progress has been achieved in the treatment of cardiovascular disease. This progress has been possible not only because of the advancement of therapeutic intervention in the disease mechanisms, but also through the early identification of patients at risk of developing the disease. Indeed, patient risk identification and early treatment are important features of modern medical practice. Over the last twenty years, a variety of factors and clinical parameters have been identified which correlate with either the current state or the future probability of developing cardiovascular disease. Such risk factors may include measurable biochemical or physiological parameters, e.g., serum cholesterol, HDL, LDL, fibrinogen levels, etc., or behavioral of life-style patterns, such as obesity, smoking, etc. (For further information see: “Cardiovascular risk factors in the elderly’, Kannel W., Coronary Artery Disease, 8:565-575, 1997 and references cited therein.) The risk factor most germane to the present invention is the level of C-reactive protein.

The intrinsic relationship between a measurable parameter or risk factor and the disease state is not always clear. In other words, it is not always clear whether the risk factor itself is causative or contributory to the disease or is instead an ancillary reflection that is indicative of the disease. Thus, a therapeutic modality, which effects a risk factor, may be directly modifying a pathological mechanism of the disease and its future course, or may be indirectly benefiting some contributory process related to the disease.

Additionally, many risk factors associated with cardiovascular disease are involved in other pathological states in either a causative or indicative role. Therefore, reduction or blockade of a particular risk factor in cardiovascular disease may have other beneficial effects in other diseases related to that risk factor.

Of particular interest to the methods of the present invention is the reduction of cardiovascular risk factors associated with abnormally high levels of C-reactive protein.

C-reactive protein is produced by the liver in response to cytokine production. Cytokines are produced as part of an inflammatory response in the body. Thus, C-reactive protein levels are a marker of systemic inflammatory activity. Chronic inflammation is thought to be one of the underlying and sustaining pathologies, in cardiovascular disease.

At menopause, with the loss of estrogen, women's prevalence of cardiovascular disease increases. Also, the risk factors of cardiovascular disease increase, especially lipid (cholesterol and triglyceride), homocysteine, and C-reactive protein levels. Today, the most common method of preventing cardiovascular disease in post-menopausal women is Hormone Replacement Therapy (HRT). However, many women do not comply with this therapy because of the unpleasant side-effects, such as bloating, resumption of mensus, breast tenderness, fear of uterine and breast cancer, etc. Additionally, while HRT does lower cholesterol and homocysteine levels, HRT raises C-reactive protein levels. A new therapeutic agent which lowers these risk factors would be beneficial.

The present invention provides tools, molecules and methods for decreasing levels of C-reactive protein in humans comprising administering to a human in need thereof an effective amount of a compound containing at least a molecule which binds C-reactive protein or a pharmaceutical salt or solvate thereof.

Further, the present invention relates to a method for inhibiting conditions or detrimental effects caused by an excess of C-reactive protein or active CRP, respectively comprising administering to a human in need thereof, an effective amount of a compound containing of at least a compound which binds C-reactive protein or a pharmaceutical salt or solvate thereof.

The present invention is based to the finding that compounds that bind CRP, i.e., antibodies, a recombinant antibody (as e.g. single chain antibody—scAb or scFv; bispecific antibody, diabody), monoclonal antibodies, are useful for lowering the levels of C-reactive protein or blocking CRP.

As used herein, the term “effective amount” means an amount of a compound which binds C-reactive protein which is capable of decreasing levels or blocking C-reactive protein and/or inhibiting conditions or detrimental effects caused by an excess of C-reactive protein or active CRP, respectively.

The term “estrogen deficient” refers to a condition, either naturally occurring or clinically induced, where a woman cannot produce sufficient estrogenic hormones to maintain estrogen dependent functions, e.g., menses,. homeostasis of bone mass, neuronal function, cardiovascular condition, etc. Such estrogen deficient situations arise from, but are not limited to, menopause and surgical or chemical ovarectomy, including its functional equivalent, e.g., medication with GnRH agonists or antagonists, ICI 182780, and the like.

The term “inhibiting” in the context of inhibiting conditions or detrimental effects caused by an excess of C-reactive protein includes its generally accepted meaning, i.e., blocking, prohibiting, restraining, alleviating, ameliorating, slowing, stopping, or reversing the progression or severity of an increase of C-reactive protein and the pathological sequelae, i.e., symptoms, resulting from that event.

The term “pharmaceutical” when used herein as an adjective, means substantially non-toxic and substantially non-deleterious to the recipient.

By “pharmaceutical formulation” or “medicament” or “pharmaceutical composition” it is further meant that the carrier, solvent, excipients and salt must be compatible with the active ingredient of the formulation (a compound of at least a molecule, which binds C-reactive protein).

The term “solvate” represents an aggregate that comprises one or more molecules of the solute, with one or more molecules of a pharmaceutical solvent, such as water, buffer, physiological salt solution, and the like.

The present invention claims a compound comprising at least a structural entity which binds C-reactive protein (CRP) or parts of it or CRP in its monomeric, pentameric or multimeric form, preferably human CRP and which
a.) blocks one or more CRP functions on cell surfaces or in a solution, preferably blood or other body fluids or from tissues, most preferably in vivo,
b.) and/or depletes CRP from a solution, preferably blood or other body fluids or from tissues, most preferably in vivo.

The term “structural entity” is readily understood by the person skilled in the art. It means a moiety within a compound which moiety is able to recognise in a specific manner at least parts of the CRP. Typically such entities can be found in antibodies or fragments thereof binding to CRP. Fragments are in particular Fab, Fv, scFv fragments. Other biomolecules such as nucleic acids or polysaccharides may also bind to CRP as well as low molecular substances which can be designed by molecular modelling techniques.

In one embodiment the compound of the invention is a polypeptide comprising a binding site to CRP, preferably an antibody containing an antigen-binding site to CRP. The compound of the invention is in particular a poly- or monoclonal antibody comprising an antigen-binding site to CRP.

The monoclonal antibody comprises particularly an antigen-binding site to CRP and is obtainable after immunising vertebrates, preferably mammals such as mice, rats, guinea pigs, hamsters, monkeys, pigs, goats, chicken, cows, horses and rabbits. The poly- or monoclonal antibody comprising an antigen-binding site to CRP is preferably humanised according to technologies well-known to the skilled person. The compound of the invention can also be prepared by immunising immune defective mice (as e.g. SCID or nude mice) repopulated with vital immune cells (e.g. of human origin; as e.g. SCID-hu mice).

In a further embodiment the antibody of the invention is a recombinant antibody (as e.g. single chain antibody—scAb or scFv; bispecific antibody, diabody etc.) capable of binding to CRP, in particular by containing the antigen-binding site of an antibody which is cross-reactive with CRP. Th antibody molecule of the invention is a humanised or human antibody. Subject matter of the invention is also a host cell, preferably a stable host cell, producing the compound of the invention.

Furthermore, subject matter of the invention is at least one recombinant vector comprising the nucleotide sequences encoding the binding molecule fragments according to the invention, operably linked to regulating sequences capable of expressing the antibody molecule in a host cell, preferably as a secretory protein.

Subject matter of the present invention is also a host comprising, preferably stably transgenic, the vector according to the invention, a prokaryotic or eukaryotic cell line producing a recombinant antibody of the invention as well as a eukaryotic organism, most preferably an animal, a plant or a fungus, producing a recombinant antibody according to the invention.

Subject matter of the invention is also a method of producing a recombinant molecule of the invention capable of binding to the CRP antigen, comprising culturing a host cell and isolating the binding molecule from the culture medium and/or the producing cell.

In another embodiment, the present invention is related with a method for inhibiting immunologic, inflammatory and/or pathophysiological responses by treating patients with increased CRP levels with the CRP-binding molecules according to the invention.

Another subject of the present invention is a pharmaceutical composition for reducing the CRP concentration, containing a therapeutically effective amount of the binding molecule according to the invention and a pharmaceutically acceptable carrier.

Still another embodiment of the invention is a method for reducing inflammatory immune and/or patho-physiological responses by reducing the CRP concentration, a method for reducing endothel injury and/or destruction by reducing the CRP concentration, a method for acute treatments in case of acute endothelial injury and/or destruction, preferably for stroke, cardiac infarction, avoidance of sudden cardiac death, for burnt offering, for severe surgery or other injuries with severe wound areas, for diabetic shock, for acute liver failure, for pancreatitis, neurodegenerative diseases, for leucaemic persons after irradiation, a method for continuous treatments in case of long term endothelial injury and/or destruction, preferably for patients with medium CRP-amounts, with atherosclerosis, with unstable angina, with diabetes type I or type II, with overweigt and/or obesity, for alcoholics, under Hormone Replacement Therapy (HRT), for old persons, for smokers, a method for preventing allograft transplant rejection or xeno-transplant rejection, a method for the induction of allo-transplant or xeno-transplant tolerance or inhibition of T cell activation, and a method for preventing or treatment of autoimmune diseases, the methods comprising administering to a patient in need of such treatment a therapeutically effective amount of a pharmaceutical composition of the invention.

The compound of the invention can be combined with other molecules, preferably therapeutics for the respective disease or other anti-inflammatory molecules like e.g. anti-IL-6-molecules, anti-IL-113-molecules. and/or complement blockers.

In a further embodiment of the invention the autoimmune disease is selected from the group consisting of diabetes mellitus, rheumatoid arthritis, multiple sclerosis, myasthenia gravis, systemic lupus erythematosis, psoriasis vulgaris, myasthenia gravis, Graves' disease, Goodpastures' disease, idiopathic thrombocytopenia purpura (ITP), aplastic anemia, inflammatory bile disease, idiopathic dilated cardiomyopathy (IDM) and autoimmune thyroiditis.

The methods provided by the current invention are useful in both the treatment and prevention of harmful sequelae associated with elevated levels of C-reactive protein. Since C-reactive protein serum concentration is related to levels and production of cytokines, which are especially produced in inflammatory processes, the methods of the current invention are useful in treating or preventing inflammatory events and sequelae, thereof. Such inflammatory events include, but are not limited to: arthritis (osteo and rheumatoid), arterial and venous chronic inflammation, autoimmune diseases, e.g., SLE, etc., and the like.

Methods of the current invention are useful for treating or preventing pathologic sequelae of atherosclerotic or thrombotic disease. Such pathologies include, but are not limited to stroke, circulatory insufficiency, ischemic events, myocardial infraction, pulmonary thromboembolism, stable and unstable angina, coronary artery disease, sudden death syndrome, and the like.

The present invention further contemplates the use of other currently known clinically relevant agents administered to treat the pathological conditions embodied in the present invention in combination with a compound of at least a molecule which binds C-reactive protein.

Moreover, the present invention contemplates that the compounds of at least a molecule which binds C-reactive protein are employed in either a treatment or prophylactic modality.

A preferred embodiment of the present invention is where the human to be administered a compound of the invention is female, and more preferred is when that human female is estrogen deficient.

Another preferred embodiment of the present invention is where the condition caused by an abnormally high level of C-reactive protein is cardiovascular disease, especially arteriosclerosis and thrombosis or other acute treatments in case of acute endothelial injury and/or destruction, like stroke, cardiac infarction, sudden cardiac death, burnt offering, severe surgery or other injuries with severe wound areas, diabetic shock, acute liver failure pancreatitis, leucaemic persons after irradiation or long term endothelial injury and/or destruction, like arteriosclerosis, diabetes type I or type II, overweight and/or obesity, alcoholism, Hormone Replacement Therapy (HRT), old persons, smokers.

A particularly preferred embodiment of the present invention is the use of a compound of at least a molecule which binds C-reactive protein in an estrogen deficient women, who is receiving estrogen or HRT, for the reduction of systemic or local inflammation.

Pharmaceutical formulations can be prepared by procedures known in the art, such as, for example, a compound of at least a molecule which binds C-reactive protein can be formulated with common excipients, diluents, or carriers, and formed into tablets, capsules, and the like.

Examples of excipients, diluents, and carriers that are suitable for formulation include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as agar, calcium carbonate, and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonire; and lubricants such as talc, calcium and magnesium stearate and solid polyethyl glycols. Final pharmaceutical forms may be: pills, tablets, powders, lozenges, syrups, aerosols, saches, cachets, elixirs, suspensions, emulsions, ointments, suppositories, sterile injectable solutions, or sterile packaged powders, depending on the type of excipient used.

Additionally, the compounds of at least a molecule which binds C-reactive protein are well suited to formulation as sustained release dosage forms. The formulations can also be so constituted that they release the active ingredient only or preferably in a particular part of the intestinal tract, possibly over a period of time. Such formulations would involve coatings, envelopes, or protective matrices, which may be made from polymeric substances or waxes.

The particular dosage of a compound containing molecules which bind C-reactive protein required to decrease levels of homocysteine and/or C-reactive protein according to this invention will depend upon the particular circumstances of the conditions to be treated. Considerations such as dosage, route of administration, and frequency of dosing are best decided by the attending physician. Generally, an effective minimum dose for oral or parenteral administration of a compound of molecules which bind C-reactive protein is about 1 to 20000 mg. Typically, an effective maximum dose is about 20000, 6000, or 3000 mg. Such dosages will be administered to a patient in need of treatment as often as needed to effectively decrease levels of C-reactive protein and/or inhibit conditions or detrimental effects caused by an excess of C-reactive protein.

CRP as Therapeutic Target in Myocardial Infarction

CRP concentrations are one of the strongest predictors of future cardiovascular events in apparently healthy persons and also indicative for the vulnerability of an atherosclerotic plaque to rupture. These remarkable observations have significantly highlightend the routine CRP measurements in cardiovascular disease management and accordingly have questioned for the mechanisms through which CRP predicts, with such accuracy, the development of diverse. vascular insults.

The missing link between CRP and cardiovascular disease could be sought in the production of the multifactorial vasoactive peptide, nitric oxide (NO) by the vascular endothelium. CRP, at concentrations known to predict adverse vascular effects, would profoundly downregulate the production of NO by endothelial cells in vitro. Results from such studies indicate that diminished NO bioactivity, in turn, inhibits anglogenesis, an important compensatory mechanism in chronic ischemia. Further, by decreasing NO synthesis, CRP may facilitate the development of diverse cardiovascular diseases. Risk reduction strategies designed to lower CRP may be effective by improving NO bioavailability. In this assay, lowering CRP concentrations by use of CRP blocking therapeutics should restore NO production.

CRP is a potent activator of the classical complement pathway, and based on clinical and experimental observations a possible role of CRP in enhancing tissue injury has been widely discussed. This concept highlights the pathogenetic importance of CRP binding to nonirremediably damaged cells leading to complement activation, which would opsonize the target cells and/or cause direct cytotoxicity, thereby increasing the amount of cell death as well as adding to the enhanced necrosis in the zone of direct ischemic necrosis. Experiments in rabbit or rat ischemic heart after coronary ligation could show that rabbit or human CRP enhances the infarct size. In vivo complement depletion could completely abrogate this effect. These studies demonstrate that human CRP and complement activation are major mediators of ischemic myocardial injury and identify them as therapeutic targets in coronary heart disease.

That CRP might be involved directly in the pathogenesis of ischemic syndroms through a proinflammatory effect mediated by complement activation could also primarily be evident by the studies of autopsy specimens showing CRP colocalized with activated complement components in infarcted myocardial tissue but not in normal myocardium. Studies carried out in a rabbit model of ischemia/reperfusion injury could show that increased plasma CRP is associated with increased myocardial tissue injury. This increase in myocardial injury results by a complement-dependent mechanism that could be ameliorated by pretreatment with complement inhibitors or prevented in rabbits deficient in complement component C6 and unable to form the cell membrane damaging complex.

The beneficial effects of CRP blocking/binding therapeutics could be shown in this rabbit model. While increasing concentrations of CRP will enhance infarct size, treatment with CRP blocking therapeutics should inhibit this enhancement in a dose dependent manner.

Another experiment can be performed with human cells. Interaction between human endothelial cells and immune cells is studied in the adhesion assay.

Adhesion of leukocytes to endothelial cells is altered with increasing concentrations of CRP. This alteration should be reversed by addition of CRP blocking molecules. Both experiments will show the beneficial effect of CRP blocking therapeutics.

Thus, CRP is a mediator and amplifier of injury secondary to ischemia. Pharmacological modulation of the plasma CRP concentration and thus repression of complement activation is an appropriate therapeutic target for the management of patients with unstable acute coronary syndrome.

1. A compound comprising at least a structural entity which binds C-reactive protein (CRP) or parts of it or CRP in its monomeric, pentameric or multimeric form, preferably human CRP and which
a) blocks one or more CRP functions on cell surfaces or in a solution, preferably blood or other body fluids or from tissues, most preferably in vivo,
b) and/or depletes CRP from a solution, preferably blood or other body fluids or from tissues, most preferably in vivo.

2. A host cell producing the compound according to claim 1.

3. A method of producing a compound according to claim 1 capable of binding to the CRP antigen, comprising the step of culturing a host cell and isolating the compound from the culture medium or the producing cell.

4. Use of a compound according to claim 1 for the manufacturing of a medicament for inhibiting immunologic, inflammatory and/or patho-physiological responses of patients with increased CRP levels.

5. A pharmaceutical composition for reducing the CRP concentration, comprising a therapeutically effective amount of the compound according to claim 1 and a pharmaceutically acceptable carrier.

6. Use of the compound according to claim 1 for the manufacturing of a medicament for reducing endothel injury and/or destruction.

7. Use of the compound according to claim 1 for the manufacturing of a medicament for acute treatments in case of acute endothelial injury and/or destruction, preferably for stroke, cardiac infarction, avoidance of sudden cardiac death, for burnt offering, for severe surgery or other injuries with severe wound areas, for diabetic shock, for acute liver failure, for pancreatitis, neurodegenerative diseases, for leucaemic persons after irradiation.

8. Use of the compound according to claim 1 for the manufacturing of a medicament for continuous treatments in case of long term endothelial injury and/or destruction, preferably for patients with medium CRP-amounts, with atherosclerosis, with unstable angina, with diabetes type I or type II, with overweigt and/or obesity, for alcoholics, under Hormone Replacement Therapy (HRT), for old persons, for smokers.

9. Use of the compound according to claim 1 for the manufacturing of a medicament for preventing allograft transplant rejection or xeno-transplant rejection.

10. Use of the compound according to claim 1 for the manufacturing of a medicament for the induction of allo-transplant or xeno-transplant tolerance or inhibition of T cell activation.

11. Use of the compound according to claim 1 for the manufacturing of a medicament for preventing or treatment of autoimmune diseases.
